(19) United States
(12) Patent Application Publication
(10) Pub. No.: US 2010/0286729 A1
(43) Pub. Date: Nov. 11, 2010

(54) DECOMPRESSION TRACTION DEVICE AND METHOD

(76) Inventors: Matthew William Haugen, St. Francis, MN (US); Jack William Smith, Minnetonka, MN (US)

Correspondence Address: Antonio Papageorgiou, Esq., Papageorgiou & Associates, 3050 Fairfield Avenue #3G, Bronx, NY, 10463, US

(21) Appl. No.: 12/774,245

(22) Filed: May 5, 2010

Related U.S. Application Data
(60) Provisional application No. 61/215,499, filed on May 6, 2009.

(51) Int. Cl. A61F 5/00

(52) U.S. Cl. 606/240

(57) ABSTRACT

A device and method is disclosed for removing or otherwise ameliorating tension from the human body. The device having a body (20) a top or cusp (22) a bottom (24) and at least one inclined surface (30). Whereby a person lays on a flat surface supporting their head and neck on the body (20) for a period of time therewith decompressing tension from their body.

### RELATED APPLICATIONS

This application claims the benefit of U.S. (Provisional) Application No. 61/215,499, filed May 6, 2009, which is hereby incorporated herein by reference.

### BACKGROUND OF THE INVENTION

This application relates generally to a device for relieving tension from the human body.

Over the past three decades there has been an increased popularity in individuals visiting chiropractors and massage therapists for their services. These practitioners can bring about relief for their patients by hands on manipulation of the human body. As the popularity of these kinds of therapies has increased over the years so have various self help home therapies. Self help home therapies, however, are limited when it comes to hands on manipulation of the human body. To assist a person in this regard, a number of devices have been proposed in an attempt to assist a person in manipulating their own body. Some examples of such devices are exercise balls to assist stretching the body, hand manipulated canes to assist massaging the body, foot operated rollers, and various forms of vibrators to reduce stress. These devices, however, are difficult to use, cause operator fatigue, and only produce localized results. Primarily, the above mentioned devices do not allow the operator to relax, which at time may cause more tension and stress in the human body than what they resolve.

Accordingly, there is a need for a device to remove or otherwise reduce tension and stress from the human body that is easy-to-use.

### SUMMARY OF THE INVENTION

The present application generally provides an apparatus and method of use to remove tension and stress from the human body that is simple and easy to use.

There are many objects of the present device disclosed herein in its various embodiments that may be addressed individually or in combinations and permutations. Each embodiment may address one or several of the following objectives.

An object of at least one embodiment of the devices disclosed herein is to provide a device for safely removing tension and stress from the human body.

Another object of at least one embodiment of the devices disclosed herein is to provide a device for easily removing tension and stress from the human body.

Another object of at least one embodiment of the devices disclosed herein is to provide a device for quickly removing tension and stress from the human body.

Another object of at least one embodiment of the devices disclosed herein is to provide a device easy to transport and store.

Another object of at least one embodiment of the devices disclosed herein is to provide a device for economically removing tension and stress from the human body.

These and other objects and advantages of the invention will be clear in view of the following description to the invention including the associated drawings.

One or more of the objects discussed herein are achieved with an apparatus for relieving tension from the human body, the apparatus that includes a body made from any rigid or semi rigid material somewhat cubic in nature having a left side and a right side.

In at least one embodiment the apparatus includes a body made from any rigid or semi rigid material somewhat cylindrical in nature having a left side and a right side.

In at least one embodiment the apparatus has a bottom that sets on a flat horizontal surface and at least one inclined surface elevating to a cusp, a first notch accommodating the cervical vertebrae and a second notch cradling the Atlas/Access C1, C2 and occipital protuberance.

In at least one embodiment the apparatus has a third notch which allows for a slight back tipping of the body, enhancing traction and decompression for the user, the body becoming slightly imbalanced from the horizontal surface by the weight of the user acting on the body.

### BRIEF DESCRIPTION OF THE FIGURES

The device of the present application will be described hereafter in detail with particular reference to the drawings. Throughout this description, like elements, in whatever embodiment described, refer to common elements wherever referred to and referenced by the same reference number. The characteristics, attributes, functions, interrelations ascribed to a particular element in one location apply to that element when referred to by the same reference number in another location unless specifically stated otherwise. All Figures are drawn for ease of explanation of the basic teachings of the present invention only; the extensions of the Figures with respect to number, position, relationship, and dimensions of the parts to form the preferred embodiment will be explained or will be within the skill of the art after the following description has been read and understood. Further, the exact dimensions and dimensional proportions to conform to specific force, weight, strength and similar requirements will likewise be within the skill of the art after the following description has been read and understood.

FIG. 1 is a perspective view of a decompression traction device.

FIG. 2 is a side elevation view of the device of FIG. 1.

FIG. 3 is a plan view of FIG. 1.

FIG. 4 is a perspective view of the device in use.

FIG. 5 is another embodiment of the device.

### DRAWINGS REFERENCE NUMERALS

| | |
|---|---|
| 20 | body |
| 22 | cusp |
| 24 | bottom |
| 26 | left side |
| 28 | right side |
| 30 | inclined surface |
| 32 | first notch |
| 34 | second notch |
| 36 | third notch |

### DETAILED DESCRIPTION OF THE INVENTION

Preferred embodiments of the invention will now be described, by way of example only and not to limit the invention, with reference to the accompanying drawings.

The present application generally provides decompression traction devices that in at least one embodiment when used relieves or at least reduces bodily tension and conditions, such as headaches. The decompression traction device further induces and releases pressure as well as providing gentle traction to the human body.

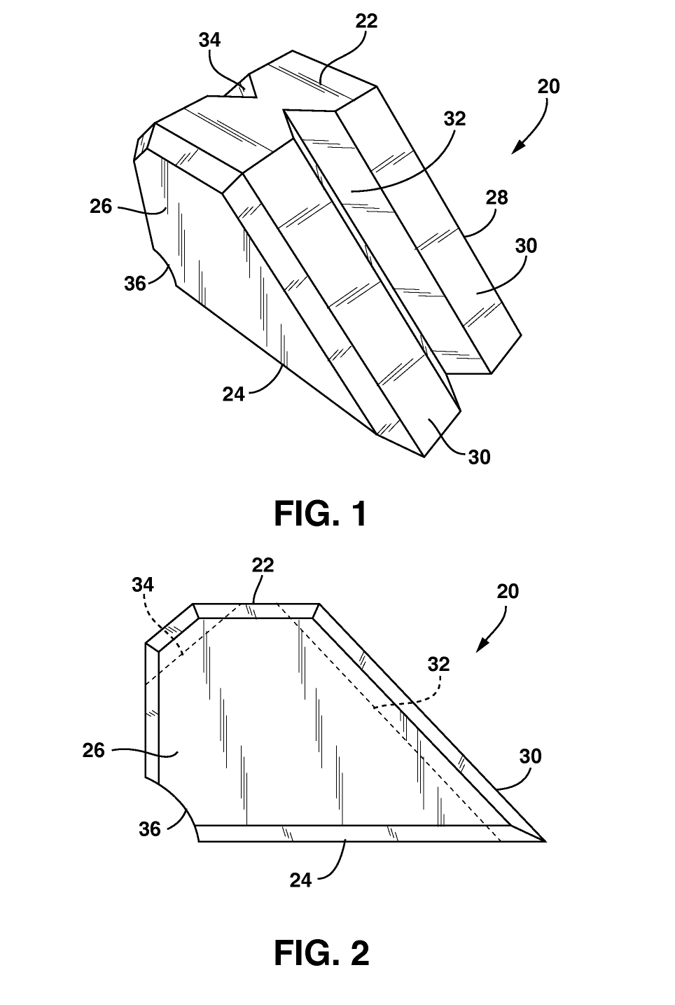

Referring to FIG. 1, a decompression traction device according to one embodiment generally has purposely shaped body 20. The device may be made from any material, preferably a rigid material or semi-rigid material. In one embodiment a semi-rigid two pound closed cell foam material is used to create the body 20. The body 20 is somewhat cubic in nature having a left side 26 and at right side 28 opposite the left side. The body 20 has a bottom 24 and a top side or cusp 22 opposite the bottom 24. Of importance the body 20 includes at least one inclined surface 30. As shown, all sides of the of the body 20 may be essentially perpendicular to each other except that the inclined surface 30 is at an angle relative to the bottom that is less than 90 degrees thereby forming a wedge shape. In this regard, the inclined surface 30 is at an angle relative to the top surface or cusp 22 that is greater than 90 degrees.

In at least one embodiment, a first notch 32 is taken from or otherwise formed into the inclined surface 30. The notch 32 is generally a groove that extends in a direction along the length of the inclined surface 30. The notch 32 may be essentially perpendicular to the lines created by the intersection of the sides 24, 28 with the inclined surface 30 as shown.

In at least one embodiment, the body 20 includes a second inclined surface between the cusp 22 and the rear side of the body 20. A second notch 34 may similarly be positioned on the second notch 34 opposite first notch 32. That is, the second notch 34 may extend in a direction along the length of the second inclined surface. Each of the notches 32, 34 may split the respective inclined surface in half or otherwise. The perimeter of the sides 24, 28 may include an at least partially beveled perimeter as shown.

FIG. 2 is a side elevation view of the device of FIG. 1. Referring to FIG. 2, the body 20 may further includes a third notch 36. In this instance, the notch 36 extends from the left side 24 to the right side 28. The purpose of third notch 36 is to subtract material from the body 20 thus diminishing the size of the bottom 24. As can be seen, the notches 32, 34 are disposed in a direction so that they are at an angle to each other, e.g., at right angles to each other. The notches may be any shape and size including circular, angular, etc.

In the preferred embodiment the dimensions of the invention are 6.75 inches long by 3.75 inches in height by 4.125 inches in width. The notches 32 and 34 are 1.0 inch in width and 0.5 inches in depth from the outer surfaces of the device. The notch 32 is 5.38 inches long at its deepest point from the outer surface of the device. The notch 34 is 1.88 inches at its deepest point from the outer surface of the device. The notch 36 is 1.0 inch in height by 1.0 inch in width by 4.125 inches in length. The chamfers shown on the outer corners of the device are 0.25 inch chamfers. The scope of the dimensions could be in a range of plus or minus 10 percent of these dimensions.

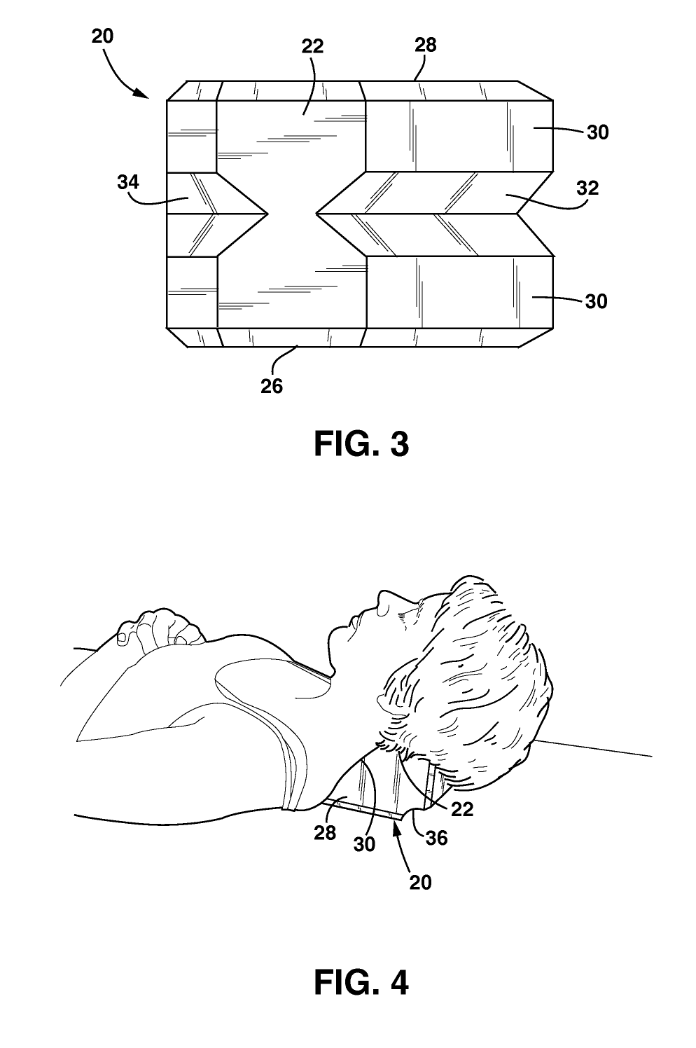

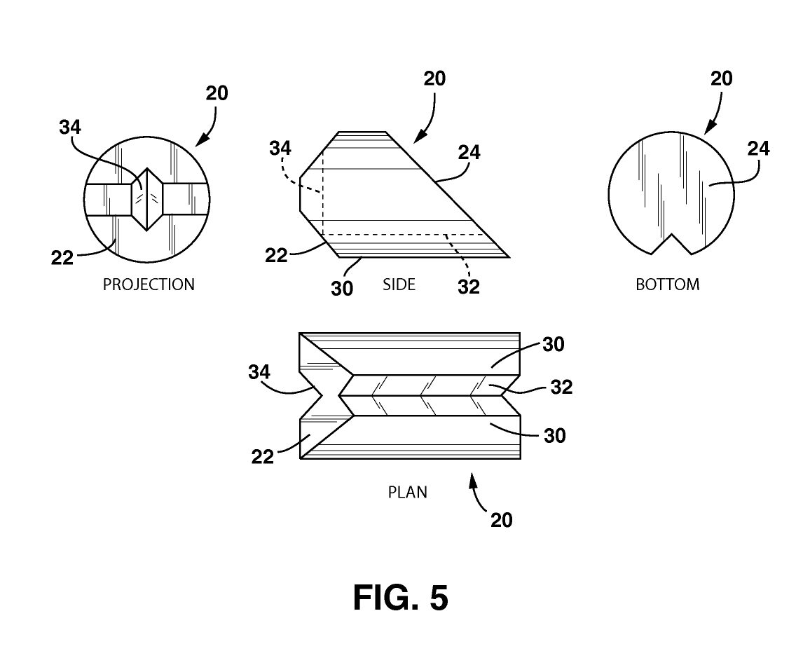

FIG. 3 is a plan view of FIG. 1 further showing the components of the design. FIG. 4 is a perspective view of the device in use. FIG. 5 is another embodiment of the device. In this embodiment of the device body 20 is cylindrical in nature.

The device generally provides pressure at the contact points with the subject's using the subject's own head weight. Additionally or alternatively, the combined design and pressure also provides a gentle and sustained traction to the cervical vertebrae. Referring to FIG. 4, the intended placement of the device is under the cervical vertebrae and the connecting point is at the sub occipital ridge at the base of the skull. The occipital ridge of the head rests on the cusp 22 of the device and hooks over resting on the back side of body 20. This position then creates a gentle traction. Boney protuberances of the cervical vertebrae are cradled in first notch 32 and second notch 34 cradles the Atlas/Access C1, C2 and occipital protuberance. Third notch 36 allows for a slight back tipping of the body 20 enhancing traction and decompression. The device can be used on any firm and stable surface like a firm bed or couch however the optimal use is on a carpeted floor. The user lies on the device for 10 to 20 minutes. The affected tissues/systems include to varying degrees, the sub occipital muscle group, transversospinalis group, upper trapezius, splenius capitus, levator scapulae, cervical vertebrae, occipital plate, occipitalis muscle and spinal fluid and more. Tension headaches, sinus headaches, migraine headaches are eased and the body is retrained to relax with regular daily use of the device.

The device of the present application provides a number of advantages. An advantage of at least one embodiment or variant of the devices disclosed herein is to provide a device for safely removing tension and stress from the human body. Another advantage of at least one embodiment or variant of the devices disclosed herein is to provide a device for easily removing tension and stress from the human body. Another advantage of at least one embodiment or variant of the devices disclosed herein is to provide a device for quickly removing tension and stress from the human body. Another advantage of at least one embodiment or variant of the devices disclosed herein is to provide a device easy to transport and store. Another advantage of at least one embodiment or variant of the devices disclosed herein is to provide a device for economically removing stress and tension from the human body. From the description above, a number of advantages of some embodiments of the decompression traction device and method become evident.

There are many materials and configurations that can be used in constructing the invention by those skilled in the art including various materials, colors and dimensions. In addition, it is clear that an almost infinite number of minor variations to the form and function of the disclosed invention could be made and also still be within the scope of the invention. Consequently, this disclosure is not intended to limit the invention to any specific embodiments and variants of the devices disclosed herein.

What is claimed is:

1. A traction device comprising a body, the body comprising a bottom side, a top side opposite the bottom side, and a first inclined surface disposed between the bottom side and the tops side therewith giving the body a wedge shape, the first inclined surface having a first notch disposed therein that extends from the bottom side to the top side of the body.

2. The traction device of claim 1, the body further comprising a second inclined surface disposed between the top of the body and a back side of the body, the second inclined surface having a second notch disposed therein that extends from the top side to the back side of the body.

3. The traction device of claim 1, wherein body has a generally cubic shape with a left side and a right side opposite the left side, each of the sides having a planar surface essentially perpendicular to the surface of the other side.

4. The traction device of claims 1, each of the bottom and the top sides each having a planer surface essentially perpendicular to the surface of the other side.

5. The traction device of claim 1, wherein the body includes a third notch disposed between the bottom side and a back side of the body, the third notch transverse in relation to the first notch.

6. The traction device of claim 1, the body further comprising a second inclined surface disposed between the top of the body and a back side of the body, the second inclined surface having a second notch disposed therein that extends from the top side to the back side of the body.

7. The traction device of claim 6, wherein the first inclined surface is oriented orthogonally with respect to the second inclined surface.

8. The traction device of claim 1, wherein the notch has a V shape with an essentially constant depth for the length of the notch.

9. The traction device of claim 1, wherein the first notch splits the inclined surface in half lengthwise.

10. A traction device comprising a body, the body comprising:
a bottom side,
a top side opposite the bottom side, each of the bottom and the top sides each having a planer surface essentially perpendicular to the surface of the other side,
a first inclined surface disposed between the bottom side and the tops side therewith giving the body a wedge shape, the first inclined surface having a first notch disposed therein that extends from the bottom side to the top side of the body,
a second inclined surface disposed between the top side and a back side of the body, the second inclined surface having a second notch disposed therein that extends from the top side to the back side of the body,
a left side,
a right side opposite the left side, the body having a third notch disposed between the bottom side and a back side of the body, the third notch transverse in relation to at least one of the first notch and the second notch.

11. The traction device of claim 10, wherein the left side and the right side each have a planar surface essentially perpendicular to the surface of the other side.

12. The traction device of claim 10, wherein the first inclined surface is oriented orthogonally with respect to the second inclined surface.

13. The traction device of claim 10, wherein the notch has a V shape with an essentially constant depth for the length of the notch.

14. The traction device of claim 10, wherein the first notch and the second notch each splits the respective inclined surface in half lengthwise.
